(19) United States
(12) Patent Application Publication
(10) Pub. No.: US 2015/0005725 A1
(43) Pub. Date: Jan. 1, 2015

(54) Quilted-Type Urinal

(71) Applicant: Mary Elizabeth Goulet, McLean, VA (US)

(72) Inventor: Mary Elizabeth Goulet, McLean, VA (US)

(21) Appl. No.: 14/044,213

(22) Filed: Oct. 2, 2013

Related U.S. Application Data
(60) Provisional application No. 61/839,630, filed on Jun. 26, 2013.

(51) Int. Cl. A61F 5/453

(52) U.S. Cl. CPC A61F 5/453
USPC 604/349

(57) ABSTRACT

A wearable quilted-type urinal for men, especially for men with mobility problems, comprises an absorbent tube including an elasticized section (such as an elasticized section resembling a wristband) at the tube's open end.

### FIELD OF THE INVENTION

The invention relates to urine collection, especially urine collection for men with mobility limitations.

### BACKGROUND OF THE INVENTION

Many adults, for various reasons, require other solutions for urine collection besides the usual approach of toilet usage. Some devices and products are commercially available to begin to address these needs.

For example, Medline's translucent portable urinal with handle, SKU: MED80235EACH, is a portable hard-plastic urinal with an attached lid, intended to be held or hung from the bedside. It is marked, graduated to 32 oz/1000 ml.

JMP's male urine guard, absorbent incontinence pouch is advertized to hold 2 oz of urine and at its closed end resembles a flat mailing envelope.

Also the patent literature includes:

U.S. Pat. No. 4,475,910 issued Oct. 9, 1984 to Conway et al. (Mentor Corp.) for “Male condom catheter having adhesive transfer on roller portion”;

U.S. Pat. No. 4,878,901 issued Nov. 7, 1989 to Sachse, for “Condom catheter, a urethral catheter for the prevention of ascending infections”;

U.S. Pat. No. RE33,206 issued May 1, 1990 to Conway, et al. (Mentor Corp.), for “Male condom catheter having adhesive on rolled portion”;

U.S. Pat. No. 5,618,277 issued Apr. 8, 1998 to Goulter, for “Condom catheter with improved valve and retaining means”;

U.S. Pat. No. 5,797,890 issued Aug. 25, 1998 to Goulter et al. (Goulter Medical Corp.), for “Support devices for retaining a male urinary incontinence condom catheter onto a penis”;

US 20020026163 published Feb. 28, 2002 by Grundke, for “Male incontinence device, application tool, and methods of assembly”;

US 20040176746 published Sep. 9, 2004 by Forral, for “Urine collection system for males utilizing a flexible external catheter and vacuum assistance”;

US 20040230180 published Nov. 18, 2004 by Goulter, for “Method and apparatus for preparing and fitting condom catheters”;

US 20050075615 published Apr. 7, 2005 by Bonham, for “Convenient urology undergarment pant system for urine collection”;

US 20050177133 published Aug. 11, 2005 by Nielsen et al., for “External urinary catheter”;

US 20050251100 published Nov. 10, 2005 by Charles, for “Close coupled urine collection chamber”;

US 20050256467 published Nov. 17, 2005 by Conley for “Adult male disposable incontinence system for disposable underwear”;

US 20060004332 published Jan. 5, 2006 by Marx for “Vacuum installed external male catheters and methods of using same”;

US 20060079854 published Apr. 13, 2006 by Kay et al. (BioDerm Inc.), for “Urine collection assembly”;

US 20070005031 published Jan. 4, 2007 by Charles, for “Incontinence collection device”;

US 20070225667 published Sep. 27, 2007 by Otto, for “Urine collection receptacle”;

US 20070225668 published Sep. 27, 2007 by Otto, for “Urine collection bag”;

US 20080077099 published Mar. 27, 2008 by House, for “External catheter with antiseptic agent”;

US 20080091155 published Apr. 17, 2008 by Matter, for “Condom catheter”;

US 20080097619 published Apr. 24, 2008 by Rioux, for “Draining bodily fluid”;

US 20090048570 published Feb. 19, 2009 by Jensen (Coloplast A/S), for “External urinary catheter”;

US 20090234310 published Sep. 17, 2009 by Marshall (Mary Marshall Enterprises, Inc.), for “Methods and devices for concealing and securing a urine collection bag”;

US 20090287170 published Nov. 19, 2009 by Otto (Preferred Medical Devices, Inc.), for “Urine collection system”;

US 201100092928 published Apr. 21, 2011 by Saze, for “Assembly for collecting urine from a male”;

US 20110152802 published Jun. 23, 2011 by DiCamillo et al., for “Incontinence diaper/underwear system”;

US 20120029451 published Feb. 2, 2012, by Conway, for “Male external urinary incontinence device”;

US 20130041352 published Feb. 14, 2013, by Smith (Xennovate Medical LLC), for “Self-cleansing bladder draining catheter”.

However, many urine collection needs are unsolved by the existing technology, and improvements on the existing technology would be desirable.

### SUMMARY OF THE INVENTION

The invention provides wearable urinals for men (especially men with mobility problems).

In a preferred embodiment, the invention provides a urinal wearable by a male, comprising: an absorbent tube having a closed end and an open end; and at least one elastic ring disposed shower-cap style or bonnet style near the open end; such as, e.g., urinals further comprising a tube-shaped waterproof exterior, wherein the absorbent tube is disposed inside the tube-shaped waterproof exterior; urinals wherein the absorbent tube comprises a hydrogel material; urinals wherein the absorbent tube comprises a quilted material; urinals wherein the absorbent tube comprises sides which are of a first thickness, and a closed end which is thicker than the first thickness; urinals wherein the absorbent tube has a diameter and a length to be worn by an adult man; urinals wherein the absorbent tube comprises a super absorbent polymer; urinals consisting essentially of the absorbent tube and the at least one elastic ring; urinals consisting essentially of the absorbent tube, the at least one elastic ring, and the tube-shaped waterproof exterior; urinals wherein urine that has been evacuated by the male wearer of the urinal is collected and contained by the urinal; urinals wherein while the man wears the urinal in bed, no urine evacuated by the man reaches the bedclothes or mattress; urinals wherein at least 12 ounces of urine are absorbed by the absorbent tube; urinals wherein at least 24 ounces of urine are absorbed by the absorbent tube; etc.

In another preferred embodiment, the invention provides a urinal wearable by a male, comprising: an absorbent tube having a closed end and an open end, wherein at the open end the absorbent tube comprises an elasticized section (such as, e.g., an elasticized section that extends at least one inch along the absorbent tube; an elasticized section that extends at least two inches along the absorbent tube, etc.); urinals wherein a floor section of the absorbent tube has a different thickness than a sidewall section of the absorbent tube; etc.

The invention in another preferred embodiment provides a wearable urinal comprising an absorbent tube having a base section of a thicker thickness than a sidewall thickness.

In another preferred embodiment, the invention provides a urinal wearable by a male, comprising: an open ended section where an elasticized tube section is disposed, wherein the elasticized tube section extends downwards at least 1 inch towards a closed end, such as, e.g., urinals comprising an absorbent material that holds at least 12 oz of urine; etc.

### BRIEF DESCRIPTION OF THE DRAWINGS

FIG. 1 is a perspective view of a wearable urinal 1 in an embodiment of the invention.

FIG. 2 is a perspective view of a wearable urinal 10 in another embodiment of the invention.

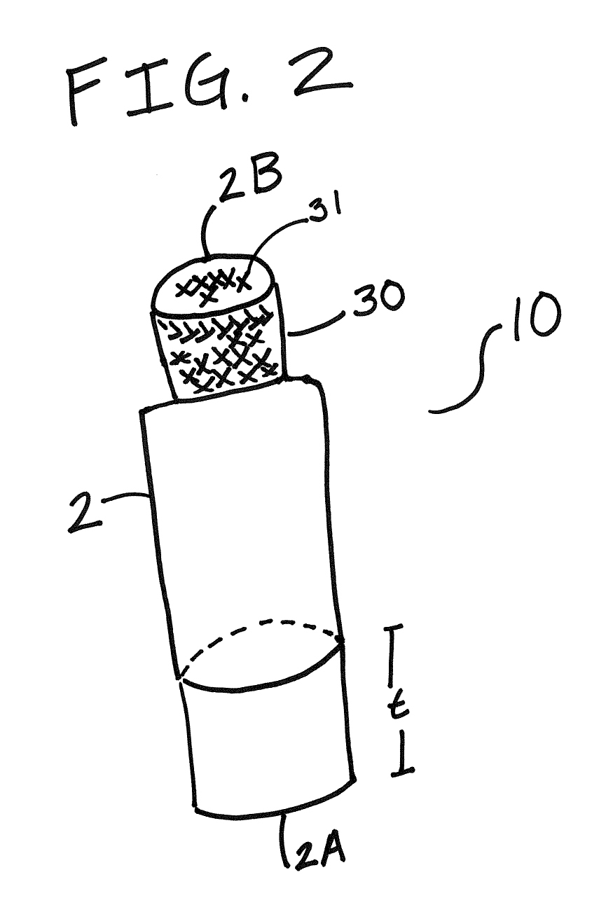

### DETAIL DESCRIPTION OF A PREFERRED EMBODIMENT

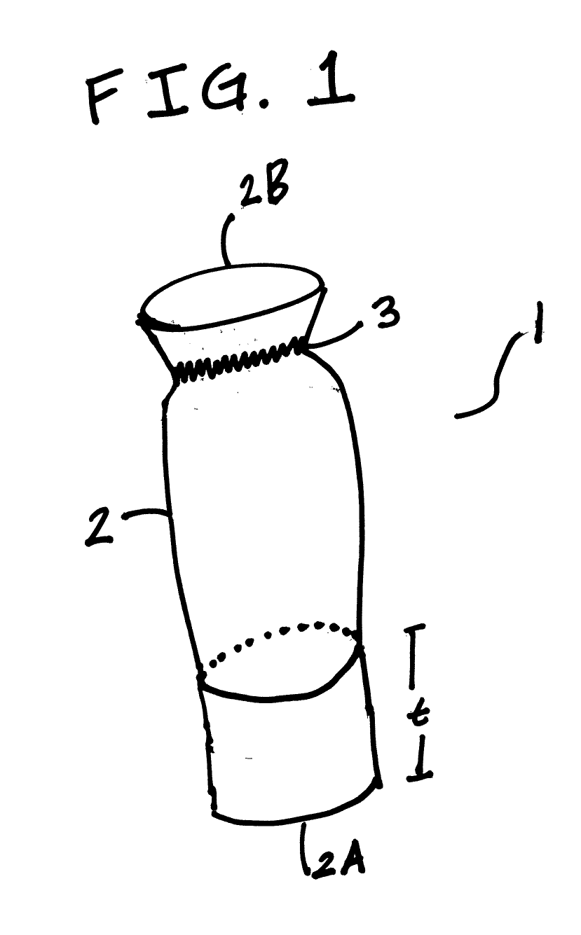

Referring to FIG. 1, a wearable urinal 1 comprises an absorbent tube 2 having closed end 2A and open end 2B. The wearable urinal 1 comprises at least one elastic ring 3 disposed shower-cap style or bonnet-style near the open end 2B. When the urinal 1 is worn by a man, the elastic ring 3 cooperates to keep to the urinal 1 from being dislodged.

The closed end 2A of urinal 1 has a thickness t which preferably is thicker than a thickness of sidewalls of the absorbent tube 2.

Although a cylindrical shape is illustrated in FIG. 1, the absorbent tube 2 may be modified to have a non-tube shape.

Examples of a man wearing urinal 1 are, e.g., wearing urinal 1 while sleeping; wearing urinal 1 while bedridden; wearing urinal 1 during waking hours (especially by a man with mobility limitations); etc.

Examples of removal and disposal of urinal 1 are, e.g., removal and disposal of urinal 1 by the wearer himself (such as in his home); removal and disposal of urinal 1 with assistance of a home health aide or other household member; hospital staff removing and disposing of urinal 1; etc.

The invention may further be appreciated in the following examples, without the invention being limited thereto.

### EXAMPLE 1

A urinal wearable by a male (preferably an adult male), comprising: an absorbent tube having a closed end and an open end; and at least one elastic ring disposed shower-cap style or bonnet style near the open end.

### EXAMPLE 1A

The urinal of Example 1, further comprising a tube-shaped waterproof exterior. The absorbent tube is disposed inside the tube-shaped waterproof exterior.

### EXAMPLE 1B

The urinal of Example 1, in which the absorbent tube comprises a hydrogel material and/or a superabsorbent polymer.

### EXAMPLE 1C

The urinal of Example 1, wherein the absorbent tube comprises a quilted material.

### EXAMPLE 1D

The urinal of Example 1, wherein the absorbent tube comprises sides which are of a first thickness, and the closed end has a thickness which is thicker than the first thickness.

### EXAMPLE 1E

Preferably the urinal of FIG. 1 has, as its elastic ring 3, an elasticized ring section 30 that extends at least 1 inch and comprises elastic thread 31. An example of an elasticized ring section 30 is a stretchable section resembling a wristband.

It will be appreciated that the invention is not limited to what has been mentioned explicitly herein, and the invention may be practiced with certain modifications and details.

What I claim as my invention is:

1. A urinal wearable by a male, comprising:
an absorbent tube having a closed end and an open end;
at least one elastic ring disposed shower-cap style or bonnet style near the open end.

2. The urinal of claim 1, further comprising a tube-shaped waterproof exterior, wherein the absorbent tube is disposed inside the tube-shaped waterproof exterior.

3. The urinal of claim 1, wherein the absorbent tube comprises a hydrogel material.

4. The urinal of claim 1, wherein the absorbent tube comprises a quilted material.

5. The urinal of claim 1, wherein the absorbent tube comprises sides which are of a first thickness, and a closed end which is thicker than the first thickness.

6. The urinal of claim 1, wherein the absorbent tube has a diameter and a length to be worn by an adult man.

7. The urinal of claim 1, wherein the absorbent tube comprises a superabsorbent polymer.

8. The urinal of claim 1, consisting essentially of the absorbent tube and the at least one elastic ring.

9. The urinal of claim 2, consisting essentially of the absorbent tube, the at least one elastic ring, and the tube-shaped waterproof exterior.

10. The urinal of claim 1, wherein urine that has been evacuated by the male wearer of the urinal is collected and contained by the urinal.

11. The urinal of claim 10, wherein while the man wears the urinal in bed, no urine evacuated by the man reaches the bedclothes or mattress.

12. The urinal of claim 1, wherein at least 12 ounces of urine are absorbed by the absorbent tube.

13. A urinal wearable by a male, comprising:
an absorbent tube having a closed end and an open end, wherein at the open end the absorbent tube comprises an elasticized section.

14. The urinal of claim 13, wherein at least 12 ounces of urine are absorbed by the absorbent tube.

15. The urinal of claim 13, wherein at least 24 ounces of urine are absorbed by the absorbent tube.

16. The urinal of claim 13, wherein the elasticized section at the open end of the absorbent tube extends at least one inch along the absorbent tube.

17. The urinal of claim 16, wherein the elasticized section at the open end of the absorbent tube extends at least two inches along the absorbent tube.

18. The urinal of claim 13, wherein a floor section of the absorbent tube has a different thickness than a sidewall section of the absorbent tube.

19. A urinal wearable by a male, comprising: an open ended section where an elasticized tube section is disposed, wherein the elasticized tube section extends downwards at least 1 inch towards a closed end.

20. The urinal of claim 19, comprising an absorbent material that holds at least 12 oz of urine.
